(19) United States
(12) Patent Application Publication
(10) Pub. No.: US 2025/0288636 A1
(43) Pub. Date: Sep. 18, 2025

(54) AGASTACHE RUGOSA EXOSOMES COMPOSITION FOR ENHANCING SKIN BARRIER AND ANTI-INFLAMMATION AND METHOD THEREOF

(71) Applicants: PEGAVISION CORPORATION, TAOYUAN CITY (TW); FacialBeau International Corporation, New Taipei City (TW)

(72) Inventors: I-Hong PAN, TAOYUAN CITY (TW); Kai-An CHUANG, TAOYUAN CITY (TW); Hsin-Jan YAO, TAOYUAN CITY (TW); Pei-Yin TSAI, TAOYUAN CITY (TW); Kuei-Chang LI, TAOYUAN CITY (TW)

(21) Appl. No.: 19/078,297

(22) Filed: Mar. 13, 2025

Related U.S. Application Data
(60) Provisional application No. 63/566,396, filed on Mar. 18, 2024.

(30) Foreign Application Priority Data
May 1, 2024 (TW) 113116273

(51) Int. Cl. A61K 36/532; A61K 8/14; A61K 8/9789; A61K 9/50; A61P 29/00; A61Q 17/00

(52) U.S. Cl. CPC A61K 36/532; A61K 8/14; A61K 8/9789; A61K 9/5068; A61P 29/00; A61Q 17/00

(57) ABSTRACT

The present disclosure provides a composition for enhancing skin barrier and anti-inflammation, including an exosome isolated from Agastache rugosa as an active ingredient. Also provided is a method for skin barrier-enhancing and anti-inflammatory, including administering to a subject in need thereof an effective amount of exosomes, in which the exosome is isolated from Agastache rugosa.

### CROSS-REFERENCE TO RELATED APPLICATION

This application claims priority to U.S. Provisional Application Ser. No. 63/566,396, filed on Mar. 18, 2024, and Taiwan Application Serial Number 113116273, filed on May 1, 2024, the disclosures of which are incorporated herein by reference in their entireties.

### BACKGROUND

### Field of Invention

The present invention relates to an exosome composition and method thereof. More particularly, the present invention relates to Agastache rugosa exosomes composition and method thereof for enhancing skin barrier and anti-inflammation.

### Description of Related Art

Skin tissue is mainly composed of epidermis, dermis and subcutaneous tissue. It provides the body's first barrier defense and can prevent the penetration of harmful substances such as allergens, irritants and microorganisms. The epidermis is located in the outermost layer of skin tissue. According to its multi-layered keratinocytes with different degrees of differentiation, it can be divided into stratum corneum, stratum lucidum, granular layer, spinous layer, basal layer and basement membrane. The stratum corneum is mainly for the skin barrier function and is composed of densely arranged keratinocytes. Because the stratum corneum is rich in lipid matrix, it has the function of preventing water loss. When the skin barrier is damaged due to aging or other adverse factors (including disease and ultraviolet radiation), transepidermal water loss will increase, resulting in a decrease in skin resistance function. The environmental allergens are easier to penetrate the skin and trigger immune and inflammatory responses, leading to problems such as atopic dermatitis, psoriasis, allergic diseases, rosacea, and facial eczema. In particular, the inflammatory reaction will aggravate the abnormality of the skin barrier function. Therefore, the inflammatory reaction and the skin barrier function form a vicious cycle.

Therefore, how to strengthen the skin barrier function to maintain healthy and stable skin, reduce dryness, itchiness, wrinkles caused by dry skin because of “damaged skin barrier”, and redness and heat caused by inflammation, the related art really needs to be improved.

### SUMMARY

The present disclosure provides a composition for enhancing skin barrier and anti-inflammation, comprising exosomes isolated from Agastache rugosa as an active ingredient.

In some embodiments, the exosomes are isolated from a shoot of the Agastache rugosa.

In some embodiments, an average particle size of each of the exosomes comprises a range of from 20 nanometers to 500 nanometers.

In some embodiments, a concentration of the exosomes comprises a range of from 1×10⁶ particles/mL to 1×10¹² particles/mL.

In some embodiments, the concentration of the exosomes comprises the range of from 9×10⁶ particles/mL to 3×10⁸ particles/mL.

In some embodiments, a transmembrane protein of the exosomes comprises cluster of differentiation 9 (CD9).

In some embodiments, the exosomes are prepared from following steps: obtaining a shoot of the Agastache rugosa; adding a monophosphate buffer solution to the shoot of the Agastache rugosa and then crushing the shoot of the Agastache rugosa to obtain a crude extract; filtering the crude extract to obtain a crude filtrate; and isolating the crude filtrate by tangential flow filtration to obtain the exosomes.

The present disclosure also provides a method for enhancing skin barrier and anti-inflammatory, comprising administering to a subject in need thereof an effective amount of a composition, wherein the composition comprises exosomes isolated from Agastache rugosa.

In some embodiments, a transmembrane protein of the exosomes comprises CD9.

In some embodiments, the exosomes are prepared from following steps: obtaining a shoot of the Agastache rugosa; adding a monophosphate buffer solution to the shoot of the Agastache rugosa and then crushing the shoot of the Agastache rugosa to obtain a crude extract; filtering the crude extract to obtain a crude filtrate; and isolating the crude filtrate by tangential flow filtration to obtain the exosomes.

In some embodiments, the composition is a cosmetic composition, a pharmaceutical composition or a food composition.

In some embodiments, a concentration of the exosomes comprises a range of from 1×10⁶ particles/mL to 1×10¹² particles/mL.

In some embodiments, the concentration of the exosomes comprises the range of from 9×10⁶ particles/mL to 3×10⁸ particles/mL.

In some embodiments, an average particle size of each of the exosomes comprises a range of from 20 nanometers to 500 nanometers.

In some embodiments, a transmembrane protein of the exosomes comprises cluster of differentiation 9 (CD9).

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 is a transmission electron microscope image of Agastache rugosa exosomes according to some embodiments of the present disclosure.

FIG. 2 shows a size and a concentration of the Agastache rugosa exosomes according to some embodiments of the present disclosure.

FIG. 3A is a high performance liquid chromatography (HPLC) chromatogram of hot water extract of Agastache rugosa according to some embodiments of the present disclosure.

FIG. 3B is an HPLC chromatogram of the Agastache rugosa exosomes according to some embodiments of the present disclosure.

FIG. 4 is a test of the protective activity of the Agastache rugosa exosomes on keratinocytes according to some embodiments of the present disclosure. The experimental results were statistically compared between groups using one-way ANOVA, ###p<0.0001 compared with the control group; *p<0.05, ****p<0.0001 compared with the hydrogen peroxide (H₂O₂) group, the results are expressed as mean±S.D. (N=3).

FIG. 5 is a microscopic observation of the protection of keratinocytes by the Agastache rugosa exosomes according to some embodiments of the present disclosure. Cell images were recorded at 10×10 magnification.

FIG. 6 shows that co-treatment of the Agastache rugosa exosomes and lipopolysaccharide (LPS) at different concentrations has no effect on cell viability according to some embodiments of the present disclosure.

FIG. 7 is a test of the anti-inflammatory activity of the Agastache rugosa exosomes on immune cells according to some embodiments of the present disclosure. The experimental results were statistically compared between groups using one-way ANOVA, ###p<0.0001 compared with the control group; **p<0.01, ****p<0.0001 compared with the bacterial LPS group, the results are expressed as mean±S.D. (N=3).

FIG. 8 shows the results of the skin irritation test of the Agastache rugosa exosomes according to some embodiments of the present disclosure.

FIG. 9 shows the results of the eye irritation test of the Agastache rugosa exosomes according to some embodiments of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number.

Exosomes are small and membrane-coated vesicles secreted by cells, ranging in size from about 30 to 150 nm. Exosomes are a double-membrane structure composed of proteins, lipids and nucleic acids. Exosomes participate in intercellular messaging by transporting specific proteins, nucleic acids, and low molecular weight metabolites; because exosomes have relatively low immunogenicity, tumor tropism, and targeting of innate and acquired immune cells. In recent years, exosomes have become the development trend of treatment and delivery systems for various diseases.

Theoretically, any cell can secrete exosomes. Exosomes from animal and human stem cells have been proven to have multiple functions such as promoting tissue regeneration, anti-aging, and immune regulation. Related research has also gradually expanded from medical functions to medical aesthetics, skin care products and other fields. However, there are no clear standards and specifications for animal stem cell exosomes at the regulatory level and skin care product application level.

Plants also secrete exosomes, called plant-derived exosome-like nanoparticles (PELNs), which are nanoscale vesicles secreted by plant cells, and contain substances such as DNA, small RNA (sRNA), microRNA (miRNA), and proteins. Since plant exosomes are of natural origin, can be mass-produced, have low immunogenicity, do not carry zoonotic pathogens, have higher bioavailability and potential biological activity, they are also a carrier tool for penetrating the skin. Active substances can enter the skin through skin-related glands (hair follicles, sebum, sweat glands), intercellular spaces and penetrating cells by exosomes. Therefore, plant exosomes are considered to be an affordable source of production while being safer and more compliant than animal or human stem cell exosomes. Therefore, plant exosomes becoming opportunities for the development of innovative raw materials.

As used herein, the term “Agastache rugosa exosomes” also called “Agastache rugosa exosome-like nanoparticles” or “Agastache rugosa derived exosome-like nanoparticles,” refers to nano-sized vesicles secreted from cells into the extracellular space.

In some embodiments, the Agastache rugosa exosomes are isolated from Agastache rugosa.

In some embodiments, each one of the Agastache rugosa exosomes has a particle size from 20 nm to 500 nm, for example, 25 nm, 30 nm, 35 nm, 40 nm, 45 nm, 50 nm, 60 nm, 70 nm, 80 nm, 90 nm, 100 nm, 150 nm, 200 nm, 250 nm, 300 nm, 350 nm, 400 nm, 450 nm, or any value between any two of these values.

In some embodiments, a concentration of the Agastache rugosa exosomes comprises a range of from 1×10⁶ particles/mL to 1×10¹² particles/mL, for example, 3×10⁶ particles/mL, 5×10⁶ particles/mL, 7×10⁶ particles/mL, 9×10⁶ particles/mL, 1×10⁷ particles/mL, 3×10⁷ particles/mL, 5×10⁷ particles/mL, 7×10⁷ particles/mL, 9×10⁷ particles/mL, 1×10⁸ particles/mL, 3×10⁸ particles/mL, 5×10⁸ particles/mL, 7×10⁸ particles/mL, 9×10⁸ particles/mL, 1×10⁹ particles/mL, 5×10⁹ particles/mL, 1×10¹⁰ particles/mL, 5×10¹⁰ particles/mL, 1×10¹¹ particles/mL, 5×10¹¹ particles/mL, or any value between any two of these values.

In some embodiments, the Agastache rugosa exosomes can be isolated by including, but not limited to ultracentrifugation, sucrose gradient centrifugation, microfiltration, tangential flow filtration, polymer precipitation, antibody magnetic bead separation, or a combination thereof.

In some embodiments, the Agastache rugosa exosomes can be obtained through the following methods, including: obtaining a shoot of Agastache rugosa; adding phosphate buffer solution to the shoot of Agastache rugosa and then crushing them to obtain a crude extract; filtering the crude extract to obtain a crude filtrate; isolating the crude filtrate by tangential flow filtration to obtain the Agastache rugosa exosomes.

In some embodiments, the Agastache rugosa exosomes are an active ingredient used for enhancing the skin barrier and have anti-inflammatory effects.

In some embodiments, the present disclosure provides a composition for enhancing skin barrier and anti-inflammation.

In some embodiments, the composition is a cosmetic composition. In some embodiments, the cosmetic composition may contain a functional additive and an ingredient commonly included in a cosmetic composition. The functional additive may include, but is not limited to, a polypeptide, a polysaccharide, a water-soluble vitamin, an oil-soluble vitamin, or a combination thereof. In addition, an oil, a fat, a humectant, an emollient, a surfactant, an organic or inorganic pigment, an organic powder, a UV absorbent, an antiseptic, a sterilizer, an antioxidant, a plant extract, a pH control agent, an alcohol, a colorant, a flavor, a blood circulation promoter, a cooling agent, an antiperspirant, purified water may be further contained.

In some embodiments, the composition is a pharmaceutical composition.

In some embodiments, the pharmaceutical composition includes the Agastache rugosa exosomes as an active ingredient, administered to an individual either orally or parenterally. In some embodiments of the present disclosure, the Agastache rugosa exosomes are formulated for administration to an individual in an oral dosage form selected from the group consisting of solutions, suspensions, emulsions, powders, lozenges, pills, syrups, lozenges, tablets, chewing gum, and capsules.

In some embodiments, the pharmaceutical composition includes the Agastache rugosa exosomes as an active ingredient and pharmaceutical acceptable carriers including, but is not limited to water, alcohols, glycol, preserving agents, antioxidants, solvent, emulsifier, suspending agent, decomposer, binding agent, excipient, stabilizing agent, chelating agent, diluent, gelling agent, preservative, lubricant, absorption enhancers, active agents, humectants, odor absorbers, fragrances, pH adjusting agents, occlusive agents, emollients, thickeners, solubilizing agents, penetration enhancers, anti-irritants, colorants, propellants, surfactant, and other similar or applicable carriers for the present invention.

In some embodiments, the composition is a food composition.

In some embodiments, the food composition can be, for example, added to edible materials in the form of food additives to prepare a food product for human or animal consumption. Food composition includes, but is not limited to, general foods, health foods, beverages, nutritional supplements, dairy products or feeds, etc. In some examples of oral dosage forms, the food composition may optionally include food-acceptable carriers, excipients and/or additives. In other examples, the dosage form of the food composition may include, but is not limited to, powders, tablets, granules, suppositories, microcapsules, ampoules, liquid sprays or suppositories.

A number of examples are provided herein to elaborate the Agastache rugosa exosomes composition of the instant disclosure. However, the examples are for demonstration purpose alone, and the instant disclosure is not limited thereto.

The present disclosure provides a composition for enhancing skin barrier and anti-inflammation, comprising exosomes isolated from Agastache rugosa as an active ingredient. An average particle size of each of the Agastache rugosa exosomes ranges from 20 nanometers to 500 nanometers, and the Agastache rugosa exosomes can be isolated from Agastache rugosa. The composition containing the Agastache rugosa exosomes as an active ingredient has the function of enhancing the skin barrier and exerting anti-inflammatory effects by inhibiting the production of nitric oxide without causing side effects on the skin.

### EXAMPLE

Although a series of operations or steps are used below to describe the method disclosed herein, an order of these operations or steps should not be construed as a limitation to the present invention. For example, some operations or steps may be performed in a different order and/or other steps may be performed at the same time. In addition, all shown operations, steps and/or features are not required to be executed to implement an embodiment of the present invention. In addition, each operation or step described herein may include a plurality of sub-steps or actions.

For the sake of clarity, features and elements that are well known in the art and are not necessary for an understanding of the principles described have been omitted.

### Preparation Example 1: Method for Preparing Agastache rugosa Exosomes

The shoot of Agastache rugosa was obtained, the surface dirt was cleaned, rinsed with pure water and dried, and then 3 times the weight of phosphate buffer was added and crushed with a juicer to obtain a crude extract. The crude extract was separated to obtain a solid part and a liquid part, and the liquid part was passed through a 0.22 μm filter membrane to remove impurities and obtain a crude filtrate. The crude filtrate was concentrated and isolated through tangential flow filtration (TFF) with a 100 kilodalton (kDa) membrane to obtain Agastache rugosa exosomes or Agastache rugosa derived exosome-like nanoparticles (AGS-TFF). The appearance of the purified Agastache rugosa exosomes was confirmed with an electron microscope, and the particle size and concentration of the Agastache rugosa exosomes were analyzed with a nanoparticle size analyzer.

### Example 1: Characterization of Agastache rugosa Exosomes

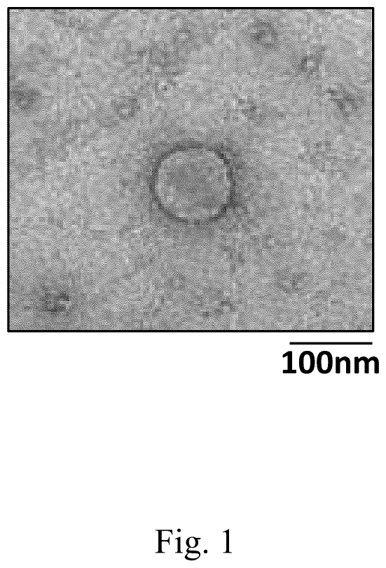

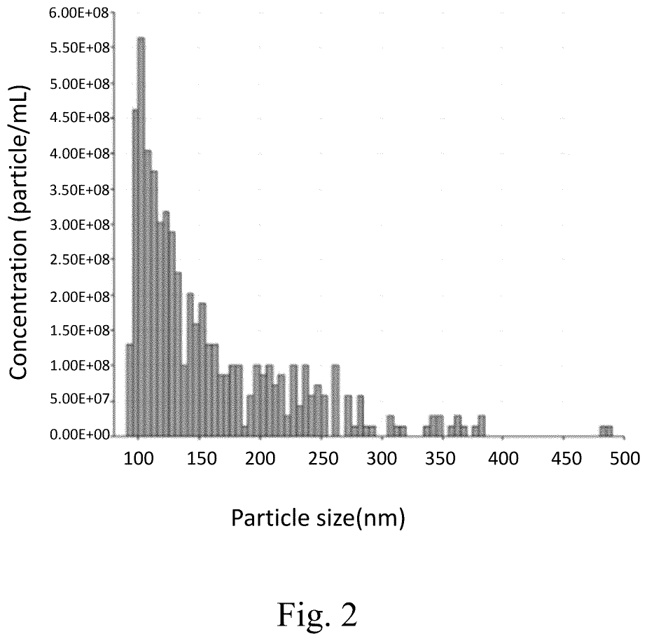

The size and properties of the Agastache rugosa exosomes obtained in Preparation Example 1 were analyzed by transmission electron microscope (TEM). FIG. 1 shows that Agastache rugosa exosomes had a diameter of 20 nanometers to 500 nanometers and were roughly spherical. The concentration of the Agastache rugosa exosomes per unit volume of 1 mL was confirmed to be 5.89×10⁹ (FIG. 2) per unit volume through the Exoid Nanoparticle Analyzer.

### Example 2: Analysis of Specific Marker CD9 Transmembrane Protein of Agastache rugosa Exosomes

Previous studies have reported that plants contain CD9 transmembrane structure similar to animal exosomes, so this example attempts to use a CD9 detection kit (ExoELISA-ULTRA Complete Kit, Cat No. EXEL-ULTRA-CD9-1, System Bioscience, UK) for marker detection. The results are shown in Table 1 below. Compared with the color values of the exosome standard, the chromogenic reaction of the Agastache rugosa exosomes obtained from Preparation Example 1 can also be seen on this platform. Therefore, it is suggested that the Agastache rugosa exosomes obtained in Preparation Example 1, in addition to the particle size and image consistent with the characteristics of exosomes, their lipid bilayer membrane also contains the common exosome-specific marker CD9 transmembrane protein.

TABLE 1

| Test sample | chromogenic reaction |
|---|---|
| (particle/mL) | value (OD₄₅₀) |
| standard sample | |
| 5.42 × 10¹⁰ | 1.47 ± 0.02 |
| 3.61 × 10¹⁰ | 1.27 ± 0.17 |
| 2.71 × 10¹⁰ | 1.31 ± 0.06 |
| 1.81 × 10¹⁰ | 0.97 ± 0.01 |
| 0.90 × 10¹⁰ | 0.57 ± 0.12 |
| 0.45 × 10¹⁰ | 0.22 ± 0.00 |
| 0.23 × 10¹⁰ | 0.12 ± 0.01 |
| 0 | 0.05 ± 0.00 |
| Agastache rugosa | |
| exosomes | |
| 2.94 × 10¹⁰ | 1.46 ± 0.09 |
| 6.54 × 10⁹ | 0.97 ± 0.08 |

### Example 3: HPLC Analysis of Agastache rugosa Exosomes

According to literature reports, generally hot water extracts of Agastache rugosa contain two main metabolites: rosmarinic acid and tilianin. In order to compare the components of Agastache rugosa extract and Agastache rugosa exosomes, HPLC was chosen to conduct HPLC component analysis of these two samples. The HPLC analysis method is described as follows:
1. Hot water extract: 10 g of the shoot of the Agastache rugosa was boiled with 100 ml water for 1 hour, then the filtrate was obtained and concentrated by vacuum concentration, and the concentrated filtrate was obtained for freeze-drying. 1 mL of deionized water was added into 25 mg of the dried sample to redissolve. The filtrate was passed through a 0.45 μm filter membrane for HPLC analysis.
2. Agastache rugosa exosomes: the Agastache rugosa exosomes obtained from Preparation Example 1 was passed through a 0.45 μm filter membrane for HPLC analysis.

HPLC analysis conditions:
Mobile phase A: 0.1% formic acid/water
Mobile phase B: 0.1% formic acid/acetonitrile

| Time (min) | Solution A (%) | Solution B (%) |
|---|---|---|
| 0 | 80 | 20 |
| 1 | 80 | 20 |
| 7 | 50 | 50 |
| 8.5 | 2 | 98 |
| 9 | 2 | 98 |
| 10 | 80 | 20 |
| 12 | 80 | 20 |

Chromatography column: ACQUITY UPLC® CSHTM C18 column (2.1*100 mm, 1.7 μm, Waters Corporation, Milford, MA, USA)
Flow rate: 0.4 mL/min, UV=330 nm
Column Temp: 35° C., Injection Volume: 2 μL

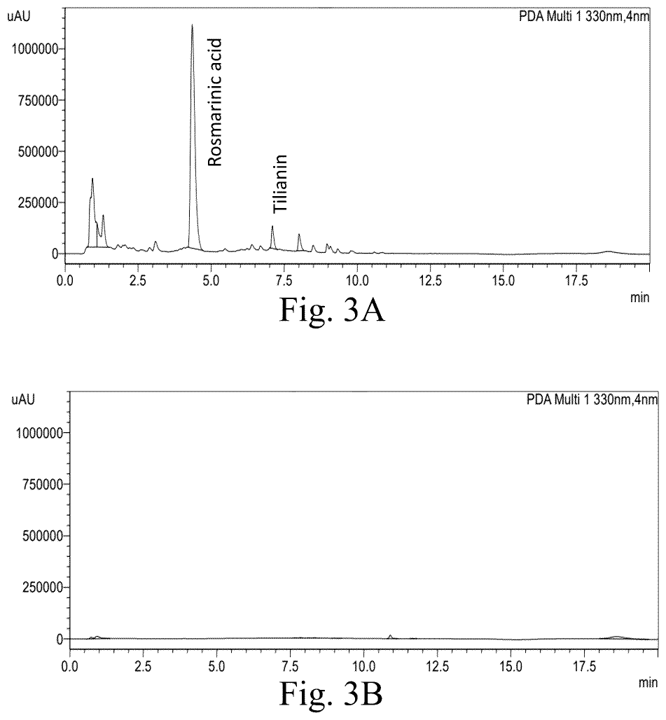

The results, as shown in FIG. 3A, indicate that the hot water extract of Agastache rugosa contains two components, rosmarinic acid and tilianin. However, as shown in FIG. 3B, the two components rosmarinic acid and tilianin were not detected in the Agastache rugosa exosomes of Preparation Example 1 at the concentration of 5.89×10⁹.

### Example 4: Protective Activity of Skin Keratinocytes

This example simulates oxidative stress with hydrogen peroxide to induce damage to human HaCaT keratinocytes, and the protective activity of the cells after adding the Agastache rugosa exosomes obtained in Preparation Example 1 was evaluated. HaCaT keratinocytes were cultured in DMEM culture medium containing 100 units/mL penicillin, 100 μg/mL streptomycin, and 10% fetal calf serum in an environment of 5% CO₂ and 37° C. First, 1×10⁴ cells were cultured in a 96-well culture plate for 24 hours. The next day, after removing the supernatant, cell culture medium containing 9.42×10⁶, 1.83×10⁷, 3.71×10⁷, 7.36×10⁷, 1.47×10⁸, 2.95×10⁸ particles/mL of the Agastache rugosa exosomes were added to react for 1 hour, and then H₂O₂ was added to a final concentration of 200 μM in an incubator for 16 hours. On the third day, cells were treated with 0.5 mg/ml of 3-(4,5-dimethylthiazol-2-yl)-2,5-diphenyltetrazolium bromide (MTT) reacted for 1 hour and then dissolved with dimethyl sulfoxide (DMSO). The cell viability of each group was expressed as the percentage of OD570 absorbance value compared with the control cell group.

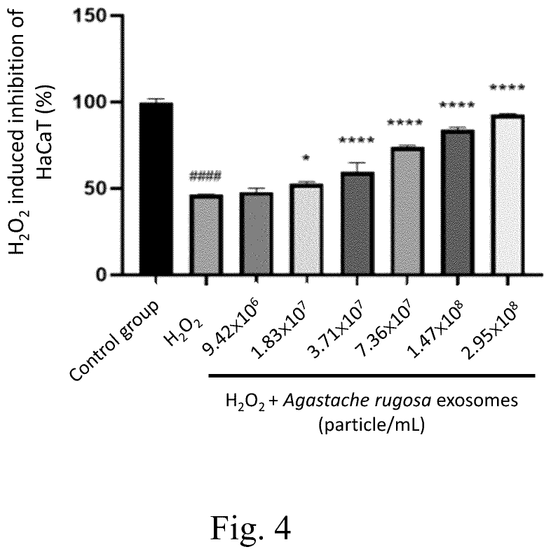

The results in FIG. 4 show that compared to the control group, H₂O₂caused a decrease in cell viability (46.3±0.3%). After adding 9.42×10⁶, 1.83×10⁷, 3.71×10⁷, 7.36×10⁷, 1.47×10⁸, and 2.95×10⁸ particles/mL of the Agastache rugosa exosomes obtained in Preparation Example 1, the cell viability returned to 47.9±2.3%, 52.8±1.1%, 59.7±5.1%, 73.9±1.0%, 83.8±1.5%, 92.8±0.5%, respectively. It indicates that the Agastache rugosa exosomes have cell protective activity in a dose-dependent manner under the platform of HaCaT damage induced by oxidative stress.

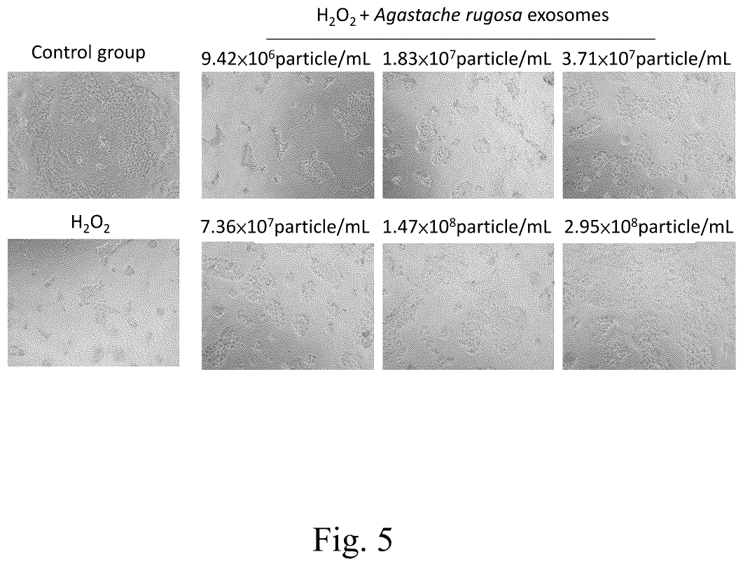

In addition, images of the Agastache rugosa exosomes protecting skin keratinocytes were also observed. The results of observing the cell morphology through a microscope showed that compared with the control group, the induction of oxidative stress H₂O₂ significantly caused the reduction of HaCaT cells. However, the results of testing the cell viability of Preparation Example 1, as shown in FIG. 5, show that the number of cells recovered with increasing concentration of the Agastache rugosa exosomes obtained in Preparation Example 1.

### Example 5: Anti-Inflammatory Activity of Immune Cells

In this example, bacterial lipopolysaccharide (LPS) was used to induce mouse macrophages RAW264.7 to produce nitric oxide (NO) free radicals to simulate an inflammatory response. The anti-inflammatory activity of the cells was evaluated after adding the Agastache rugosa exosomes obtained in Preparation Example 1. RAW264.7 immune cells were cultured in DMEM culture medium containing 100 units/mL penicillin, 100 μg/mL streptomycin, and 10% fetal calf serum in an environment of 5% CO₂ and 37° C. First, 2×10⁴ cells were cultured in a 96-well culture plate for 24 hours. The next day, after removing the supernatant, cell culture medium containing 9.42×10⁶, 1.83×10⁷, 3.71×10⁷, 7.36×10⁷, 1.47×10⁸, 2.95×10⁸ particles/mL of the Agastache rugosa exosomes were added to react for 1 hour, and then LPS was added to a final concentration of 100 nM in an incubator for 16 hours. On the third day, the supernatant was collected and the NO content was detected by Griess reagent, and the cells were reacted with MTT (0.5 mg/mL) for 30 minutes and then dissolved in DMSO. The cell viability of each group was expressed as the percentage of OD570 absorbance value compared with the control cell group.

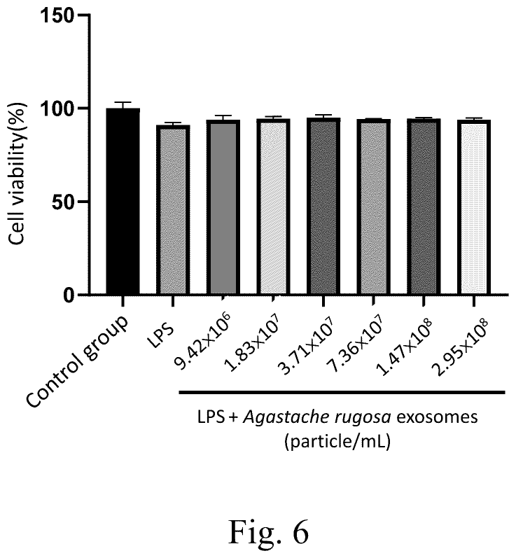

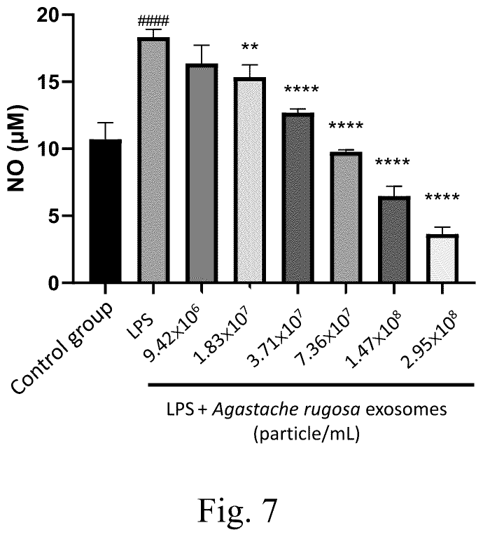

The results in FIG. 6 show that co-treatment with 0.31%, 0.63%, 1.25%, 2.5%, 5% of the Agastache rugosa exosomes obtained in Preparation Example 1 and LPS has no effect on cell viability. In the inflammatory mode (as shown in FIG. 7), LPS induced NO production (18.3±0.6 μM) compared to the control group (10.7+1.2 μM). After adding 9.42×10⁶, 1.83×10⁷, 3.71×10⁷, 7.36×10⁷, 1.47×10⁸, and 2.95×10⁸ particles/mL of the Agastache rugosa exosomes obtained in Preparation Example 1, the NO concentrations were inhibited to 16.4+1.4μM, 15.4±0.9 μM, 12.7±0.3 μM, 9.8±0.2 μM, 6.5±0.7 μM and 3.6±0.5 μM, respectively. It indicates that the Agastache rugosa exosomes obtained in Preparation Example 1 have anti-inflammatory activity in an inflammatory mode in a dose-dependent manner.

### Example 6: Safety Assessment

In this example, a safety assessment test was conducted on an in vitro bionic leather platform, and a skin irritation test was conducted on the Agastache rugosa exosomes of Preparation Example 1. Skin irritation test includes the following operations. The test was conducted on a human skin model based on the test kit (EpiSkin™ KIT, WPISKIN/S/13) approved by the Organization for Economic Co-operation and Development (OECD) TG 439. During the test, the Agastache rugosa exosomes of Preparation Example 1 were dissolved in 1×PBS to a final concentration of 5.89×10⁸ particles/mL, and then directly added topically to the surface of the skin model. After a 15-minute reaction, the Agastache rugosa exosomes was washed away, and the skin model was cultured for 42 hours. Finally, cell viability analysis (MTT assay) was used to evaluate the cell viability effect of Preparation Example 1 or positive control group (5 wt % SDS). The evaluation standard for irritation was based on the United Nations Globally Harmonized System of Classification and Labeling of Chemicals (UN GHS) Type 2. When the cell viability was ≤50%, it is considered to be skin irritating.

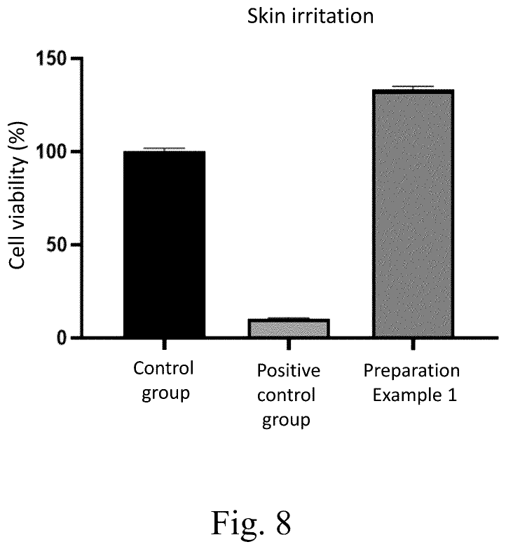

Please refer to FIG. 8 for the test results. FIG. 8 is the skin irritation test results of Preparation Example 1, the control group and the positive control group disclosed in this disclosure. Experimental data are expressed as mean±S.D., and the samples number is 3 in each group. After the skin irritation test, the viability of the control group was 100.0±1.9%, the cell viability of Preparation Example 1 was 133.2±1.8%, and the cell viability of the positive control group was 10.5±0.2%. As shown in FIG. 8, the Agastache rugosa exosomes of Preparation Example 1 show no irritation in the skin irritation test.

Furthermore, this example also conducts a safety evaluation test for eye irritation, and conducts an eye irritation test for the Agastache rugosa exosomes of Preparation Example 1. This test is conducted in accordance with OECD TG 492 specifications and uses a test kit (LabCyte™ CORNEA-MODEL, Cat. No. 411324) to test human corneal epithelial tissue model. During the test, the Agastache rugosa exosomes of Preparation Example 1 were dissolved in 1×PBS to a final concentration of 5.89×10⁸ particles/mL, then directly added to the surface of the corneal epithelial tissue model locally, and washed away after 60 minutes of reaction. The corneal epithelial tissue model was cultured for 24 hours, and finally the cell viability effect of Preparation Example 1 or positive control group (ethanol) was evaluated by cell viability analysis (WST-8 assay kit). The evaluation standard for irritation was based on UN GHS Type 2. When the cell viability was ≤40%, it is considered to be eye irritating.

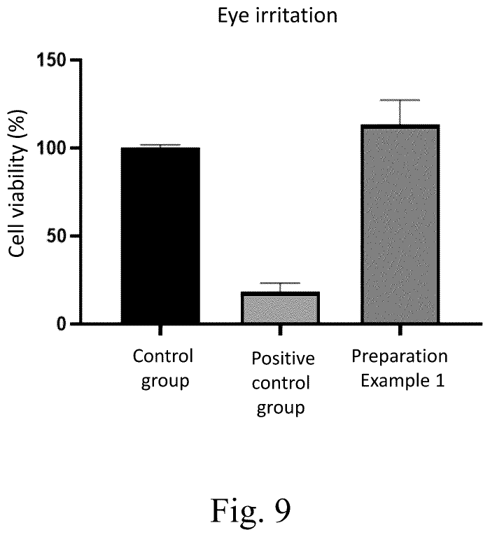

Please refer to FIG. 9 for the test results. FIG. 9 is the corneal epithelial tissue irritation test results of Preparation Example 1, the control group and the positive control group disclosed in the present disclosure. Experimental data are expressed as mean±S.D., and the samples number is 3 in each group. After the eye irritation test, as shown in FIG. 9, the cell viability of the control group was 100.0±1.8%, the cell viability of Preparation Example 1 was 113.3±13.8%, and the cell viability of the positive control group was 18.2±5.1%. As shown in FIG. 9, the Agastache rugosa exosomes of Preparation Example 1 show no irritation in the eye irritation test.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A composition for enhancing skin barrier and anti-inflammation, comprising exosomes isolated from Agastache rugosa as an active ingredient.

2. The composition of claim 1, wherein the exosomes are isolated from a shoot of the Agastache rugosa.

3. The composition of claim 1, wherein an average particle size of each of the exosomes comprises a range of from 20 nanometers to 500 nanometers.

4. The composition of claim 1, wherein a concentration of the exosomes comprises a range of from 1×10⁶ particles/mL to 1×10¹² particles/mL.

5. The composition of claim 4, wherein the concentration of the exosomes comprises the range of from 9×10⁶ particles/mL to 3×10⁸ particles/mL.

6. The composition of claim 1, wherein a transmembrane protein of the exosomes comprises CD9.

7. The composition of claim 1, wherein the exosomes are prepared from following steps:
obtaining a shoot of the Agastache rugosa;
adding a monophosphate buffer solution to the shoot of the Agastache rugosa and then crushing the shoot of the Agastache rugosa to obtain a crude extract;
filtering the crude extract to obtain a crude filtrate; and
isolating the crude filtrate by tangential flow filtration to obtain the exosomes.

8. A method for enhancing skin barrier and anti-inflammatory, comprising administering to a subject in need thereof an effective amount of a composition, wherein the composition comprises exosomes isolated from Agastache rugosa.

9. The method of claim 8, wherein a transmembrane protein of the exosomes comprises CD9.

10. The method of claim 8, wherein the exosomes are prepared from following steps:
obtaining a shoot of the Agastache rugosa;
adding a monophosphate buffer solution to the shoot of the Agastache rugosa and then crushing the shoot of the Agastache rugosa to obtain a crude extract;
filtering the crude extract to obtain a crude filtrate; and
isolating the crude filtrate by tangential flow filtration to obtain the exosomes.

11. The method of claim 8, wherein the composition is a cosmetic composition, a pharmaceutical composition or a food composition.

12. The method of claim 8, wherein a concentration of the exosomes comprises a range of from 1×10⁶ particles/mL to 1×10¹² particles/mL.

13. The method of claim 12, wherein the concentration of the exosomes comprises the range of from 9×10⁶ particles/mL to 3×10⁸ particles/mL.

14. The method of claim 8, wherein an average particle size of each of the exosomes comprises a range of from 20 nanometers to 500 nanometers.

15. The method of claim 8, wherein a transmembrane protein of the exosomes comprises CD9.
